(19) United States
(12) Patent Application Publication
(10) Pub. No.: US 2012/0022329 A1
(43) Pub. Date: Jan. 26, 2012

(54) APPARATUSES FOR ADVANCING AN ENDOSCOPE THROUGH A PASSAGE

(76) Inventors: Mihir S. Wagh, Gainesville, FL (US); Roberto Montane, JR., Gainesville, FL (US); Carl D. Crane, Gainesville, FL (US)

(21) Appl. No.: 13/262,329

(22) PCT Filed: Apr. 1, 2010
(86) PCT No.: PCT/US10/29579

§ 371 Date: Sep. 30, 2011

Related U.S. Application Data
(60) Provisional application No. 61/165,596, filed on Apr. 1, 2009.

(51) Int. Cl. A61B 1/00

(52) U.S. Cl. 600/118; 600/129

(57) ABSTRACT

In one embodiment, an endoscope includes a body, an elongated shaft that extends from the body, the elongated shaft ending in a distal tip, a first anchoring device that is fixed to the elongated shaft, and a second anchoring device that is extendable from the elongated shaft. In one embodiment, an endoscope advancing apparatus includes a first anchoring device adapted to be fixed to an elongated shaft of an endoscope, and a second anchoring device adapted to be mounted to an opposite side of the elongated shaft in a manner in which the second anchoring device can be axially extended from the elongated shaft.

### BACKGROUND

Endoscopes are now commonly used in medicine for diagnosing conditions and performing surgery. A common application for endoscopes is in the digestive system. For example, endoscopes are often passed through the mouth and esophagus and into the stomach, or through the anus into the colon. Unfortunately, it is more challenging to reach deeper parts of the digestive system, such as the small intestine. Specifically, it can be challenging to advance an endoscope through the various twists and turns of the small intestine.

### BRIEF DESCRIPTION OF THE FIGURES

The present disclosure may be better understood with reference to the following figures. In the figures, like reference numerals designate corresponding parts throughout the figures, which are not necessarily drawn to scale.

FIG. 1 is a side view of an embodiment of an endoscope.

FIG. 2 is a perspective view of a distal tip of the endoscope of FIG. 1.

FIG. 3 is a schematic side view of the distal tip of FIG. 2, illustrating internal portions of anchoring devices of the endoscope.

FIGS. 4A-4F are sequential schematic views of the endoscope of FIG. 1 being advanced through a passage using the anchoring devices identified in FIGS. 2 and 3.

FIGS. 5A and 5B illustrate endoscope advancing apparatus that can be added to an endoscopic device.

FIG. 6 is a perspective view of an embodiment of a suction head for an anchoring device.

FIG. 7 is a cross-sectional view of the suction head of FIG. 6, shown anchored to tissue.

FIG. 8 is schematic view of an embodiment of a system for automatically advancing an endoscope through a passage.

### DETAILED DESCRIPTION

Disclosed herein are apparatuses that can be used to advance an endoscope through a passage, such as a passage within the body. In some embodiments, an endoscope includes an integral apparatus that can be used to advance the endoscope through the passage. In other embodiments, independent endoscopic advancing apparatus is applied to an endoscope (in a retrofitting scenario) that itself does not contain other advancing apparatus. In either case, the apparatus includes anchoring devices that secure an endoscope in place within a passage and draw the endoscope forward through the passage. In some embodiments, the apparatus comprises a fixed suction element and an extendable and retractable suction element that are alternately used to advance the endoscope through the passage.

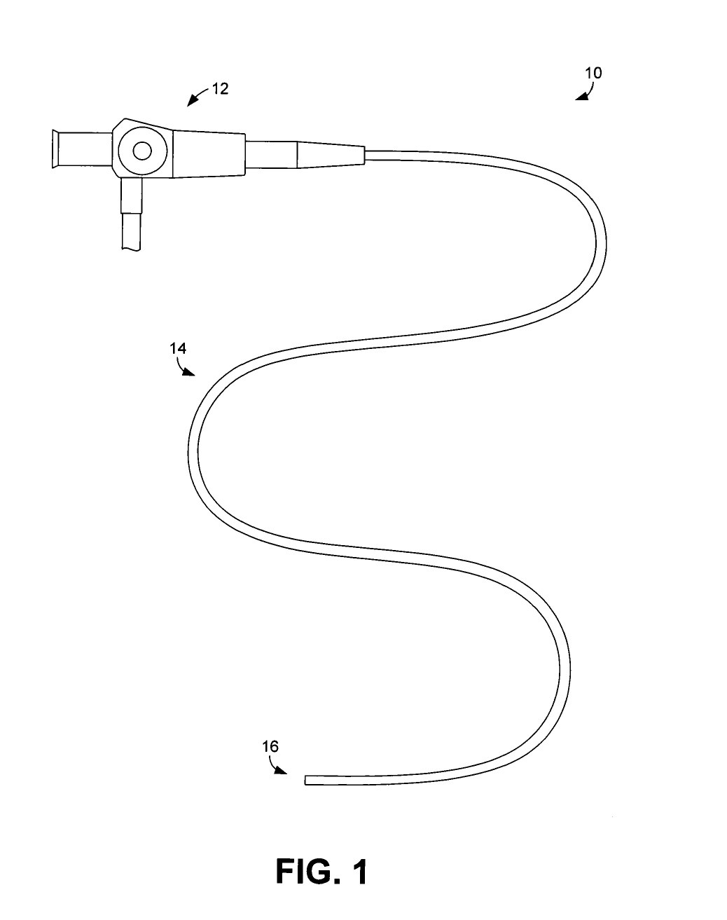

FIG. 1 illustrates an endoscope 10. The endoscope 10 includes a body 12 from which extends an elongated shaft 14 that is adapted for insertion and advancement through a passage, such as a lumen within the human body. In some embodiments, the shaft 14 is a flexible tube adapted for insertion and advancement through the small intestine. As shown in FIG. 1, the shaft 14 terminates in a distal tip 16. In some embodiments, the distal tip 16 can be manually articulated using a suitable control element provided on the endoscope body 12.

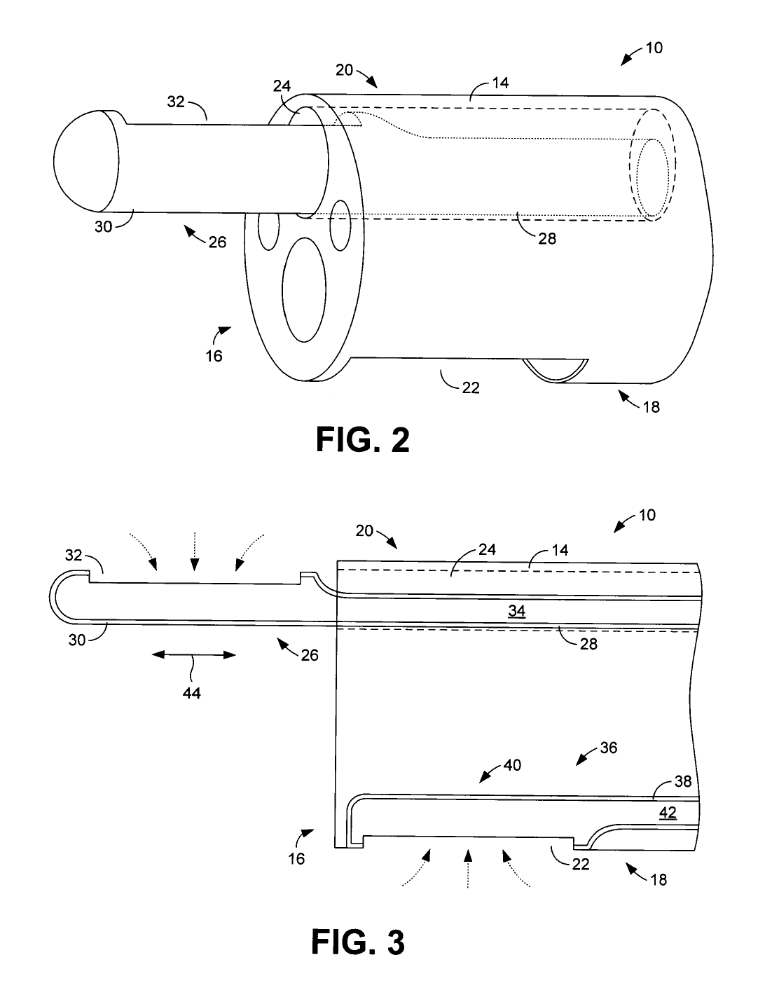

Referring next to FIG. 2, the distal tip 16 of the elongated shaft 14 is shown in greater detail. As indicated in FIG. 2, the shaft 14 is generally circular in cross-section and may be said to have a first side 18 and a second side 20, which is opposite to the first side. In the orientation of the shaft 14 shown in FIG. 2, the first side 18 can be considered the bottom side of the shaft and the second side 20 can be considered the top side of the shaft. Provided on the first side 18 of the shaft 14 adjacent the distal tip 16 is a suction port 22 that, as described below, forms part of a first anchoring device that is used to advance the endoscope 10 through a passage. Extending through the shaft 14 to the distal tip 16 is a working channel 24 in which is provided a second anchoring device 26 that is used to advance the endoscope 10 through a passage. In the embodiment of FIG. 2, the second anchoring device 26 includes an elongated tube 28 that terminates in a suction head 30 that includes a suction port 32, which is in fluid communication with an inner lumen 34 of the tube (see FIG. 3).

With reference next to FIG. 3, the first anchoring device 36, which is contained within the elongated shaft 14, is shown. As is apparent in FIG. 3, the first anchoring device 36 is similar in construction to the second anchoring device 26. Therefore, the first anchoring device 36 includes an elongated tube 38 that terminates in a suction head 40. The suction head 40 includes the suction port 22, which is in fluid communication with an inner lumen 42 of the tube 38.

Although the first anchoring device 36 is fixed relative to the elongated shaft 14, the second anchoring device 26 is extendable from and retractable within the working channel 24, as indicated by arrow 44. Accordingly, the first anchoring device 36 can be referred to as the fixed anchoring device and the second anchoring device 26 can be referred to as the movable, extendable, and/or retractable anchoring device. As described below, the suction heads 30 and 40 function to draw in tissue (see dashed arrows) for purposes of securing anchoring devices 26 and 36 to the wall of a passage through which the endoscope 10 is to be advanced.

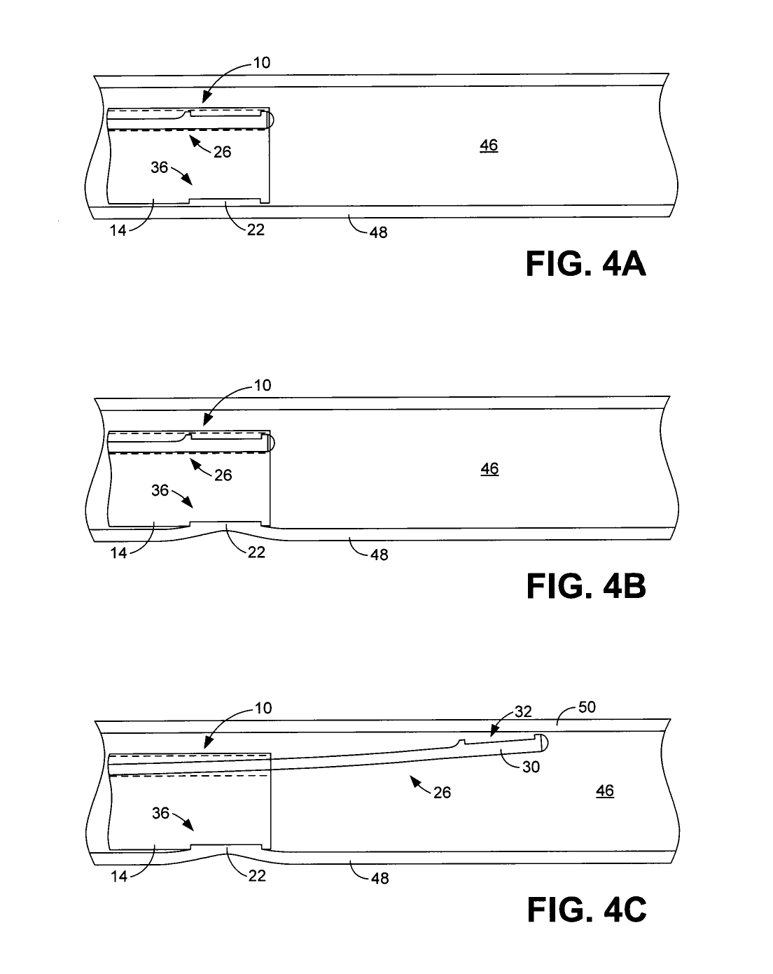

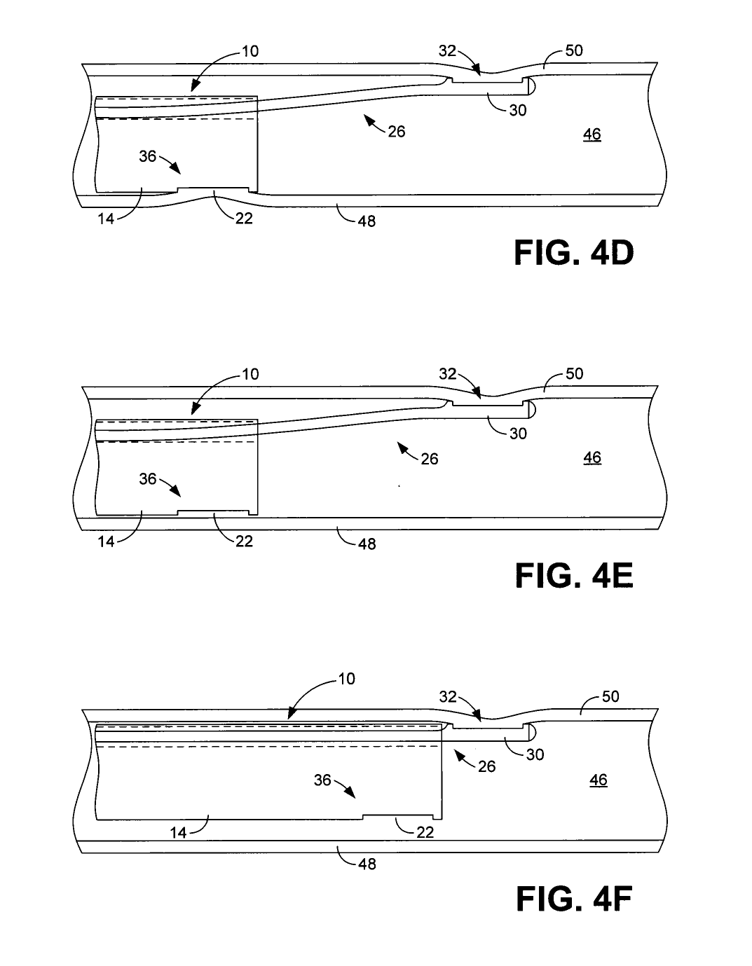

FIGS. 4A-4F depict advancement of the endoscope 10 through a passage 46. By way of example, the passage 46 comprises a lumen of the small intestine. Beginning with FIG. 4A, the endoscope 10 is introduced into the passage 46 and is manipulated so that the suction port 22 of the first anchoring device is positioned close to a wall 48 that defines the passage. Next, with reference to FIG. 4B, suction is applied to the first anchoring device so as to draw the endoscope 10 to the wall 48 and/or the draw wall to the endoscope. In cases in which the wall is flexible, a portion the wall 48 may be drawn into the suction port 22, as depicted in FIG. 4B. At this point, the endoscope 10 is anchored to the wall 48.

Once the endoscope 10 has been anchored to the wall 48 in the manner described above, the second anchoring device 26 can be extended from the endoscope, as illustrated in FIG. 4C. In particular, the second anchoring device 26 is extended, either manually or automatically, to a position distal to the endoscope tip 16 at which the anchoring device's suction head 30 is positioned adjacent to the wall 50 opposite the wall 48. In some embodiments, the second anchoring device 26 can have a natural curvature and/or bias that facilitates placement of the suction head 30 close to the wall 50.

Referring next to FIG. 4D, suction is then applied to the second anchoring device 26 so as to draw the second anchoring device to the wall 50 and/or draw the wall to the second anchoring device. In cases in which the wall 50 is flexible, a portion of the wall may be drawn into the suction port 32 of the suction head 30, as depicted in FIG. 4D. At this point, the shaft 14 is securely anchored to opposite walls 48, 50 of the passage 46.

Next, the suction applied to the first anchoring device 36 is halted to release the endoscope 10 from the wall 48, as illustrated in FIG. 4E. Optionally, a puff of air or other fluid can be used to facilitate such release. Once the endoscope 10 has been released, the second anchoring device 26 can be retracted back into the shaft 14. Because the second anchoring device 26 is still anchored to the wall 50, such “retraction” actually advances the endoscope 10 through the passage 46 toward the suction head 30 of the second anchoring device, as shown in FIG. 4F. In the case of the small intestine, the intestine may at least to some degree move toward the endoscope 10 rather than the endoscope moving toward the point at which the suction head 30 is anchored to the intestine due to the intestine's flexibility and mobility. Regardless, relative movement is achieved and the endoscope 10, in effect, advances through the passage 46 as desired.

At this point, the endoscope 10 has been advanced a given distance through the passage 46. Suction can then be applied to the first anchoring device 36 again to secure the endoscope 10 in its new position within the passage 46. To effect further advancement, the suction provided to the second anchoring device 26 can be removed to release the second anchoring device from the wall 50 and the process described above in relation to FIGS. 4B-4F can be repeated until the endoscope 10 is advanced to the desired location within the passage 46.

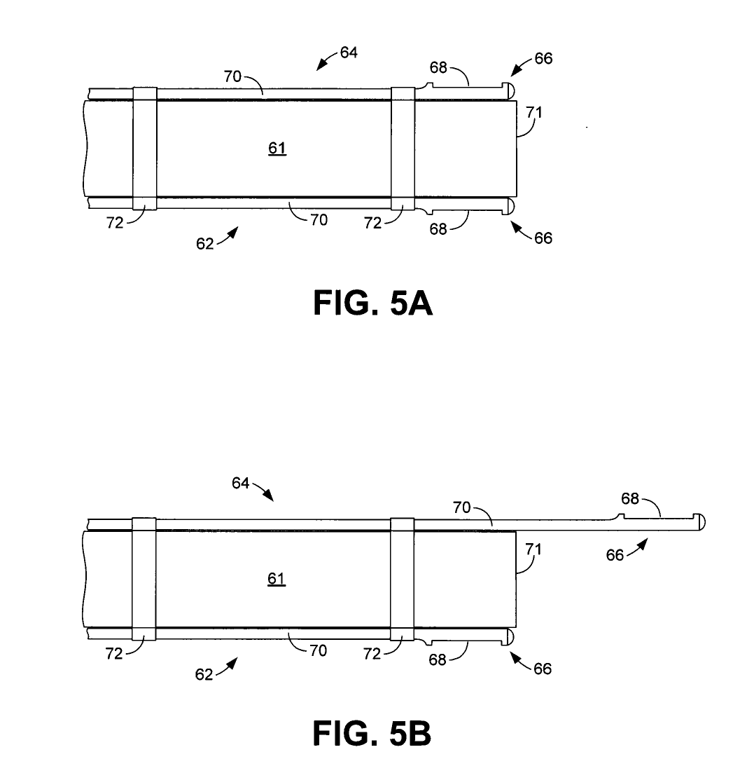

In the above-described embodiment, an endoscope included an integral advancing means and therefore can be considered to comprise a self-advancing endoscope. In other embodiments, however, a non-self-advancing endoscope can be provided with independent advancing means in order to enable similar operation. FIGS. 5A and 5B illustrate an example of one such advancing means used in conjunction with the exterior of the endoscope 60. As shown in those figures, first and second anchoring devices 62 and 64 are applied to the exterior of the endoscope 60 and its shaft 61. The anchoring devices 62, 64 each include a suction head 66 having a suction port 68 and a tube 70 that extends to the suction head. The first anchoring device 62 is not meant to move relative to the endoscope 60 and may therefore be referred to as the fixed anchoring device. The second anchoring device 64, however, is intended to be extended and retracted relative to the endoscope shaft tip 71 and may therefore be referred to as the movable, extendable, and/or retractable anchoring device. Connection of the anchoring devices 62 and 64 can be achieved in any way in which the first anchoring device 62 is securely fixed and the second anchoring device 64 can axially translate relative to the shaft 61. In some embodiments, the anchoring devices 62 and 64 are connected to the endoscope shaft 61 using biocompatible bands or clips 72.

As described above, the disclosed endoscopes can be used to traverse passages within the body, such as the small intestine. In such an application, the anchoring devices can attach to and detach from the distal mucosa of the intestine. Although the suction force that is applied is strong enough to attain the desired endoscope locomotion, the suction force is selected so as to not be so strong as to tear or otherwise damage the mucosa. For example, the suction force is selected so that the anchoring device can be forcibly separated from the mucosa with full suction applied without causing damage. In some embodiments, a suction force of approximately 2 Newtons (N) to 5 N is applied to the intestine walls.

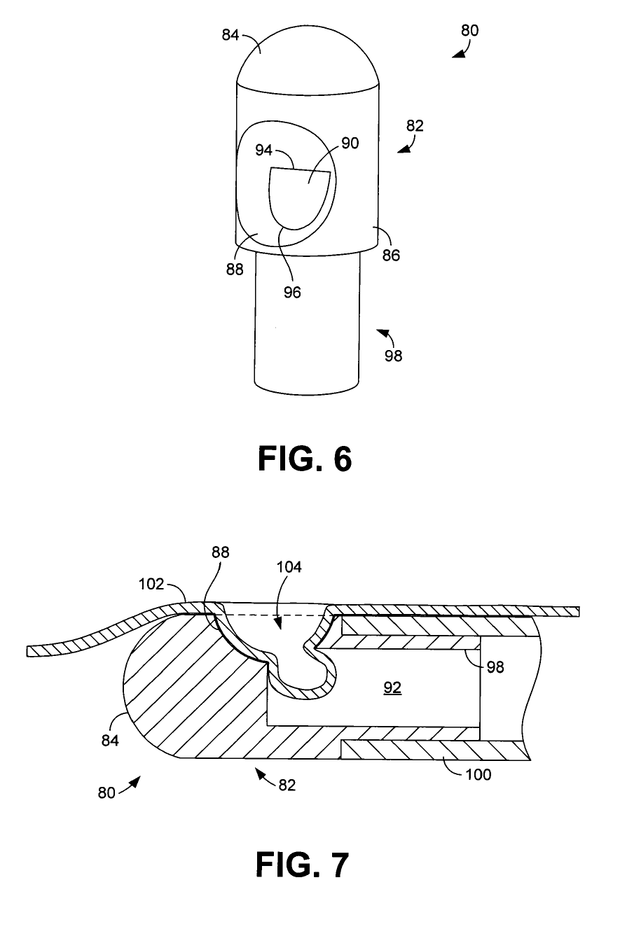

FIG. 6 illustrates an example suction head 80 that can be used to form an anchoring device similar to those described above. In the embodiment of FIG. 6, the suction head 80 comprises a main body 82 having a generally cylindrical shape. At the end of the body 82 is a rounded (e.g., hemispherical) distal tip 84 that facilitates passage of the anchoring device through a lumen. Formed in an outer surface 86 of the body 82 is a suction port that includes an indentation or depression 88. In some embodiments, the depression 88 is generally circular and comprises a concave, curved surface. In further embodiments, the depression is generally hemispherical. Formed within the depression 88 is a hole 90 that is in fluid communication with a inner passage 92 formed within the suction head 80 (see FIG. 7). In the illustrated embodiment, the hole 90 is defined by a straight edge 94 and an arcuate edge 96.

Extending from the base of the body 90 is a cylindrical mounting tube 98 that is adapted to be received by an associated tube 100 that applies suction to the suction head 80 (see FIG. 7). In some embodiments, the body 90 and the mounting tube 98 are unitarily constructed from a single piece of material, such as a metal like aluminum.

FIG. 7 illustrates use of the suction head 80. More particularly, FIG. 7 illustrates the suction head 80 being used to draw in tissue 102, such as the wall of the small intestine, into the depression 88 and the hole 90 so as to anchor the suction head to the tissue. As shown in the figure, the tissue 102 can be drawn deeply within the suction head 80 (identified by reference numeral 104) to ensure strong anchoring. Because of the flexibility and elasticity of the tissue 102, such drawing does not damage the tissue, as long as the pull force applied to the tissue is properly managed.

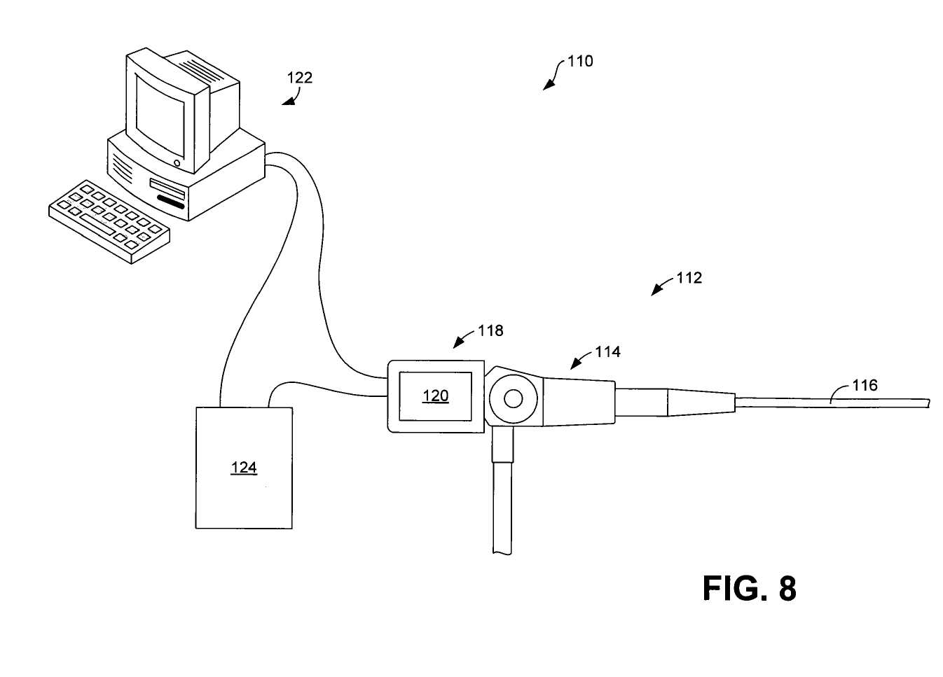

In some embodiments, advancement of an endoscope can be automated. Specifically, the apparatus used to advance the endoscope can be mechanized such that the endoscopist need not manually apply and release suction force or extend and retract an anchoring device. FIG. 8 schematically illustrates an example system 110 for automatically controlling advancement of an endoscope through a passage, such as the small intestine. As shown in FIG. 8, the system 110 includes an endoscope 112 that, similar to the endoscope 10, comprises a body 114 and a flexible shaft 116 that extends outwardly therefrom. For purposes of this discussion, it is assumed that the endoscope 112 comprises integral advancement apparatus, such as the anchoring devices 26 and 36 described above in relation to FIGS. 2 and 3. Accordingly, the advancement apparatus is not visible in FIG. 8.

The system 110 further comprises an automatic advancement unit 118 that, in this example, is mounted to the body 114 of the endoscope 112. The automatic advancement unit 118 is connected at least to the movable anchoring device of the endoscope 112 and includes a motor 120, such as a servomotor, that is adapted to extend or retract the movable anchoring device according to commands received by a control unit 122. In the illustrated embodiment, the control unit 122 comprises a desktop computer that executes appropriate control software. The system 110 also includes a dedicated suction source 124 that is controlled by the control unit 122 to provide a predetermined level of suction force to the anchoring devices of the endoscope 112. By way of example, the suction source 124 comprises one or more pumps that generate suction.

The system 110 can be used to automate advancement of the endoscope 112 during surgical procedures. Once the endoscope shaft 116 has been positioned in the desired location within a passage by the operating surgeon, the system 110 can be activated to automate advancement. The control unit 122 then sends control commands to the suction source 124 to cause it to deliver suction force to the fixed anchoring device to secure it to the passage wall in similar manner to that shown in FIG. 4B. Notably, the amount of pull force that is applied to the wall of the passage (e.g., small intestine wall) is controlled by monitoring the pressure within the fixed anchoring device and increasing or decreasing the vacuum as necessary. By way of example, the pressures can be sensed by one or more pressure sensors (not shown) that are associated with the suction source 124, the advancement unit 118, or the anchoring device, and the pressure values can be provided to the control unit 122 in a feedback loop. With that information, the control unit 122 can carefully control the pull force to be strong enough to provide for secure anchoring, but not so strong as to damage the passage wall.

Next, the control unit 122 can send control commands to the automatic advancement unit 118 to cause its motor 120 to extend the movable anchoring device from the endoscope shaft 116 similar in manner to that shown in FIG. 4C. The distance to which the movable anchoring device is extended can be monitored during such extension using a suitable detector, such as an encoder, associated with the advancement unit 118. That distance can also be provided to the control unit 122 in a feedback loop.

Once the movable anchoring device has been extended to the desired extent, the control unit 122 can halt further extension and send control commands to the suction source 124 to cause it to deliver suction force to the movable anchoring device to secure it to the passage wall in similar manner to that shown in FIG. 4D. Again, the control unit 122 can carefully control the pull force to be strong enough to provide for secure anchoring, but not so strong as to damage the passage wall. In addition, the control unit 122 can send control commands to the suction source 124 to cause it to stop delivering suction force to the fixed anchoring device to cause it to release the passage wall in similar manner to that shown in FIG. 4E.

The control unit 122 can then send control commands to the advancement unit 118 to cause its motor 120 to retract the movable anchoring device relative to the endoscope shaft 116 in similar manner to that shown in FIG. 4F. The distance to which the movable anchoring device is retracted can be monitored by the control unit 122 in similar manner to the extension.

At this point, endoscope 112 has been advanced through the passage and the process can be repeated by the system 110, if desired, to continue such advancement. It is noted that although the system 110 of FIG. 8 is shown as comprising separate components, including an automatic advancement unit 118, a control unit 122, and a suction source 124, one or more of those components can be integrated into a single device. Such a device can be coupled to an endoscope, as the unit 118 is shown coupled to the endoscope 112 in FIG. 8, or can be independent of the endoscope. In the latter case, the endoscopist can hold and operate the endoscope, while the other apparatus controls the anchoring devices and the suction they apply.

Although various medical applications have been described herein, it will be appreciated that the disclosed advancing apparatuses can be applied to endoscopic devices used in other applications, such as industrial applications.

Claimed are:

1. An endoscope comprising:
a body;
an elongated shaft that extends from the body, the elongated shaft ending in a distal tip;
a first anchoring device that is fixed to the elongated shaft; and
a second anchoring device that is extendable from the elongated shaft.

2. The endoscope of claim 1, wherein each anchoring device includes a suction head having a suction port.

3. The endoscope of claim 2, wherein each anchoring device further includes an elongated tube that includes an inner lumen that is in fluid communication with the suction port.

4. The endoscope of claim 2, wherein the suction port of the first anchoring device faces a first direction and wherein the suction port of the second anchoring device faces a second direction that is opposite to the first direction.

5. The endoscope of claim 4, wherein the first and second directions both face radially outward from the elongated shaft.

6. The endoscope of claim 1, further comprising a working channel provided within the elongated shaft, wherein the second anchoring device is provided within and extendable from the working channel.

7. An endoscope advancing apparatus comprising:
a first anchoring device adapted to be fixed to an elongated shaft of an endoscope; and
a second anchoring device adapted to be mounted to an opposite side of the elongated shaft in a manner in which the second anchoring device can be axially extended from the elongated shaft.

8. The advancing apparatus of claim 7, wherein each anchoring device includes a suction head having a suction port.

9. The advancing apparatus of claim 8, wherein each anchoring device further includes an elongated tube that includes an inner lumen that is in fluid communication with the suction port.

10. The advancing apparatus of claim 8, wherein the suction port of the first anchoring device is adapted to face a first direction and wherein the suction port of the second anchoring device is adapted to face a second direction that is opposite to the first direction.

11. The advancing apparatus of claim 10, wherein the first and second directions both face radially outward from the elongated shaft.

12. The advancing apparatus of claim 7, further comprising connection elements adapted to connect the first and second anchoring devices to the elongated shaft.

13. The advancing apparatus of claim 12, wherein the connection elements include a band or a clip.

14. A system for automatically advancing an endoscope through a passage, the system comprising:
a first anchoring device adapted to be fixed relative to the endoscope;
a second anchoring device adapted to be axially extendible and retractable relative to the endoscope;
a motor adapted to extend and retract the second anchoring device;
a suction source adapted to provide suction force to the first and second anchoring devices; and
a control unit adapted to control the motor and the suction source such that extension and retraction of the second anchoring device and the application of suction force to the first and second anchoring devices is automatically controlled by the control unit.

15. The system of claim 14, wherein the first and second anchoring devices comprise integral parts of the endoscope.

16. The system of claim 14, wherein the first and second anchoring devices are retrofitted to the endoscope.

17. The system of claim 14, wherein the motor forms part of a unit that is mounted to the endoscope.

18. The system of claim 14, further comprising a sensor that detects the pressure within at least one of the anchoring devices and wherein the control unit is adapted to use the detected pressure to control the amount of suction force that is provided by the suction source.

19. The system of claim 14, further comprising a detector that senses the distance of extension of the second anchoring device and wherein the control unit is adapted to use the sensed distance to control extension and retraction of the second anchoring device.

20. A method for advancing an endoscope through a passage, the method comprising:
(a) positioning a first anchoring device associated with the endoscope near a wall of the passage;
(b) applying suction to the first anchoring device to anchor the first anchoring device to the passage wall;
(c) extending a second anchoring device associated with the endoscope to position the second anchoring device near the passage wall at a position distal to the endoscope;
(d) applying suction to the second anchoring device to anchor the second anchoring device to the passage wall;
(e) reducing the suction applied to the first anchoring device to release it from the passage wall; and
(f) retracting the second anchoring device relative to the endoscope to cause the endoscope to advance through the passage.

21. The method of claim 20, further comprising reducing the suction applied to the second anchoring device to release it from the passage wall and then repeating steps (a)-(f) to further advance the endoscope through the passage.

22. The method of claim 20, wherein the passage is the small intestine.

23. The method of claim 20, wherein the extension and retraction of the second anchoring device and the application of suction are automatically controlled by a control unit that monitors suction force and second anchoring device position.
